(19) United States
(12) Patent Application Publication
(10) Pub. No.: US 2020/0306289 A1
(43) Pub. Date: Oct. 1, 2020

(54) WOUND HEALING TOPICAL FORMULATION AND PREPARATION THEREOF

(71) Applicant: COMSATS UNIVERSITY ISLAMABAD, Islamabad (PK)

(72) Inventors: Fazli WAHID, Abbottabad (PK); Taous KHAN, Abbottabad (PK); Naveera NAEEM, Abbottabad (PK)

(73) Assignee: COMSATS UNIVERSITY ISLAMABAD, Islamabad (PK)

(21) Appl. No.: 16/827,940

(22) Filed: Mar. 24, 2020

(30) Foreign Application Priority Data
Mar. 29, 2019 (PK) 193/2019

(51) Int. Cl. A61K 33/06; A61K 31/722; A61P 17/02

(52) U.S. Cl. CPC A61K 33/06; A61P 17/02; A61K 31/722

(57) ABSTRACT

Provided is a formulation including chitosan impregnated with a modified clay. The modified clay is a smectite clay modified with cations. The formulation is effective for healing a burn wound. It is superior in skin regeneration and patient comfort than commonly available clinical treatments. The formulation can be used for the treatment of dermal wounds including burn, incision, excision, and abrasion wounds.

### BACKGROUND

The disclosed embodiments relate to the field of tissue engineering. Herein, functional components are fabricated into chitosan by a precisely optimized protocol to form various formulations, which act as a skin substitute.

Burns are thermal traumatic injuries to the skin or other tissues, causing disruption to some or all the cells. In thermal burns, the degree of cellular damage varies depending on the duration and exposure to temperature. As the normal skin structure is disrupted, its function to maintain homeostasis is impaired, resulting in water loss and reduction in body temperature. In order to compensate heat loss, the metabolic rate is increased, and therefore body fats and proteins are consumed. Thus, burns are the most severe form of trauma causing tissue degeneration, immune suppression, hyper-metabolism and abnormal pathophysiological responses. These factors may favor microbial penetration and affect the proper healing process of skin, which necessitates an ideal treatment.

Till date, various therapeutics and pharmaceutical products have been designed to compensate the tissue loss, provide barrier and prevent infection after thermal trauma. Biological dressings like pig skin and cadavers of human skin are considered effective treatment for burn wounds but their use is limited as it elicits immune response, and thus they must be changed after 2 to 5 days. Likewise, they have finite availability and are very expensive. Similarly, the other available treatment options involve the application of Vaseline gauze to cover the wound, but this treatment is uncomfortable to the patient as it requires frequent change of dressing, which traumatizes the new tissues and slows down the healing process. In addition, gauzes are not applicable to movable parts of body. Furthermore, antimicrobial ointments are also available, which serve to control infection but pose risk of toxicity.

Therefore, the current desire is to design a product which is biodegradable, is nontoxic, is porous, adsorbs wound exudate, is non-adherent, has sustainable availability and maintains a moist environment to favor transport of growth mediators to the wound interface. Presently, ‘biomaterials’ are widely explored for their biomedical applications, as these materials mimic normal anatomical and functional properties of extra-cellular matrix (ECM). To date, biomaterials have the highest rate of success in the field of tissue engineering, where each biomaterial has associated advantages and, unfortunately, disadvantages as well. Cellulosic dressings, despite their wound healing potential and capacity for painless and non-damaging dressing change, lack bactericidal property, have poor biodegradability, and require wood consumption. Similarly, collagen or dry fibrin thrombin based wound dressings are fragile and susceptible to damage by bending and pressure. Biomaterial based hydrogels are unable to absorb the wound exudate due to their moist nature.

Therefore, there remains a need for an improved burn dressing that assists in proper healing and is advantageous to patient compliance. In this domain, chitosan finds promising applications due to its properties like biocompatibility, biodegradability along with anti-microbial and non-immunogenic properties. In addition, its biodegradable product is also a component of ECM, which facilitates in new skin tissue formation. Chitosan is a component of crustacean exoskeleton and fungal cell wall, obtained by the deacetylation of chitin in the presence of alkali.

Chitin and chitosan have therapeutic properties that are useful in various fields. For example, chitosan can be applied in powder or liquid form to stop bleeding, due to its ability to bind with red blood cells (RBCs). Chitosan based fibrous mats exhibit good attachment and proliferation of L29 cells, for example. And chitosan with alginate promotes healing of full-thickness excision wounds.

### SUMMARY

The disclosed embodiments include a novel chitosan-based tissue regenerative dressing with a bioactive component, aiming to compensate problems associated with the existing standard burn treatment and other biomaterial dressings. Here, the bioactive component is a metal modified smectite mineral, which imparts additional mechanical and biological properties to the dressing.

The dressing is a pharmaceutical product for effective healing of a burn wound. It is superior in skin regeneration and patient comfort than commonly available clinical treatments. The product can be in the form of a thin membrane prepared by an immersion precipitation phase inversion method, comprising of chitosan mainly, with the impregnation of metals modified clays. Herein, a chitosan solution was prepared in lactic acid (w/v), which was then loaded with a type of smectite clay (montmorillonite), having different modifications, to obtain functionalized membranes. Furthermore, these components can be processed into different forms, according to their use, such as gel, nanofibers, scaffold and others.

All these components have their role in ensuring proper wound healing. These embodiments provide a physical barrier to burn associated pathogens, which has the ability to clear pathogens from the wound site. The chitosan polymer activates and recruits growth factors to the wound site and provides a framework to the new epithelial cells in order to mimic the extra cellular matrix, while the clay provides additional mechanical and antimicrobial properties. Collectively, the designed composite membranes provide a favorable environment for new skin tissue regeneration. Therefore, these membranes can be used for the treatment of dermal wounds including burn, incision, excision and abrasion wounds.

### BRIEF DESCRIPTION OF THE DRAWINGS

FIG. 1 depicts a method of preparing fabricated chitosan composite membranes of the disclosed embodiments.

FIG. 2 shows how formulations of the disclosed embodiments can be used to promote healing.

FIGS. 3A and 3B demonstrate quantitatively that formulations of the disclosed embodiments have a positive effect on healing.

FIGS. 4A-4D show photographs of H&E stained tissue demonstrating that formulations of the disclosed embodiments promote excellent new skin tissue formation.

### DETAILED DESCRIPTION OF EMBODIMENTS

In burn wounds, epithelial tissues are completely lost, which require a skin substitute for wound coverage with inherent re-epithelialization property. For this purpose, various formulations have been made that accelerate wound healing and tissue regeneration. In the disclosed embodiments, chitosan is impregnated with modified clays and formulated in a film form to be used as a wound healing and tissue regenerative formulations. In addition, the components can be further processed into a number of forms like gel, nano fibers, scaffold and others with the adaptation in the protocol, to be used accordingly.

Herein, at first, a type of smectite clay—montmorillonite (MMT)—was modified with a monovalent (Na^{t}) or divalent cation (Ca²⁺ or Cu²⁺) to produce modified clay I (Na-MMT), II (Ca-MMT) and III (Cu-MMT), respectively. For the modification, an ion exchange reaction method was used, where the base in the alumino-silicate layers of MMT was exchanged with the desired ion. In addition to these ions, MMT can be modified with Ag⁺, Zn²⁺ and other functional ions.

The protocol starts with adding 10 g of the refined MMT in 100 ml of 0.2 mol/L salt solution of respective cations (NaCl, CaCl₂ and CuSO₄). After addition, the solution was stirred magnetically for 6 h at 60° C. It was then centrifuged for 15 min at a speed of 8000 g to settle down the modified clay. These sediments were washed with deionized water 3-4 times and dried completely in a drying oven at 80° C. After drying, it was ground with a pestle and mortar to a 300 mesh size.

For the preparation of modified membranes, chitosan solution was prepared. For this, 1-4% chitosan polymer (˜90% degree of deacetylation), specifically 2%, was added in 1% organic acid (lactic acid) solution. This solution was homogenously mixed by placing in a shaking incubator at 30° C. and 150-200 rpm, overnight. Undissolved particles were removed by filtering through nylon cloth. This solution can be casted to obtain a chitosan membrane or can be used for preparation of clay modified membranes.

For the preparation of modified chitosan membranes, 1-4%, precisely 2% of each finely grinded metal modified clay (Na-MMT, Ca-MMT and Cu-MMT) was magnetically stirred separately in chitosan solution, giving formulation I (chitosan-Na-MMT), II (chitosan-Ca-MMT) and III (chitosan-Cu-MMT). The membranes were prepared by casting 25-30 ml solution in glass petri plates and drying at 50-60° C. until sufficient drying was achieved. These membranes were neutralized with 10% NaOH for 30-45 min and washed several times with distilled water to remove alkali. Prepared membranes were lyophilized or stored in distilled water for later use. Similarly, the components can be electro spun to form nanofibers or freeze dried to obtain scaffolds or sponge or hydrogels.

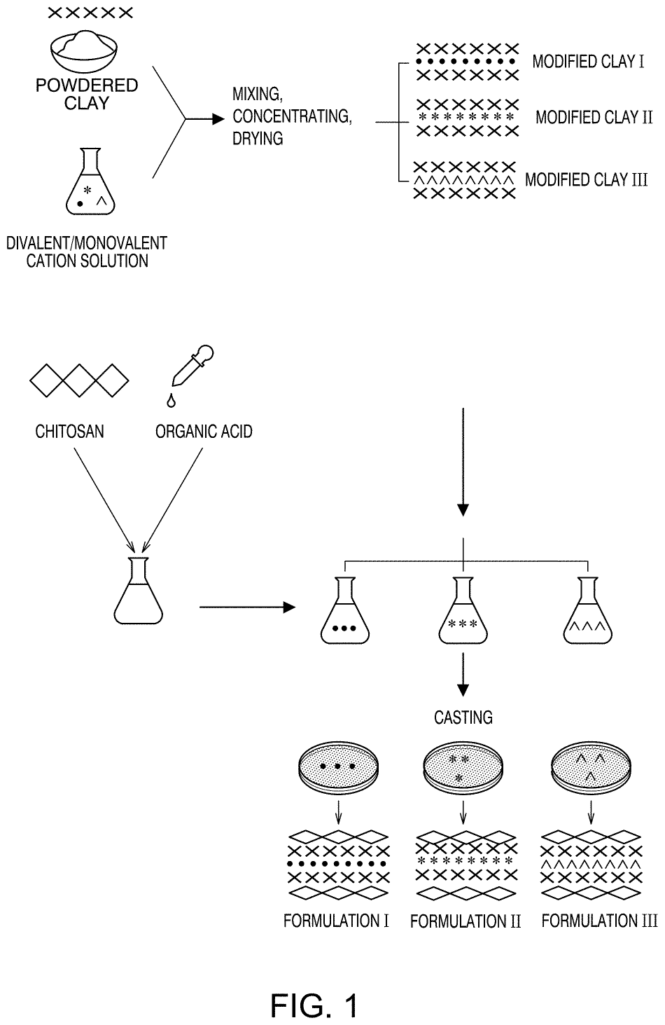

FIG. 1 depicts the preparation method of fabricated chitosan composite membranes by immersion precipitation phase inversion method. The aluminosilicate clay was modified with one of three different cations (Na⁺, Ca²⁺ and Cu²⁺) by dissolving a definite amount (10 g) of clay in 0.2 mol/L cationic solution. This solution was magnetically stirred at a suitable temperature (60° C.). The solution was then concentrated by centrifuge machine at 8000 g for 15 min. The pellet obtained was kept overnight in a drying oven at 80° C. to remove water and was finely grounded.

For the preparation of membranes, a specific amount (2% w/v) of chitosan powder was added in an organic acid (lactic acid, 1% v/v) solution and mixed uniformly. Then, 2% modified clays were added separately in chitosan solution and mixed using a magnetic stirrer. The solution was then cast into standard size glass Petri plates and dried in an oven. The membranes obtained were immersed in alkaline solution (10% NaOH) for precipitation and washed with distilled water. The prepared membranes can give an ideal environment to the wound bed to effectively heal with the prevention of microbial invasion.

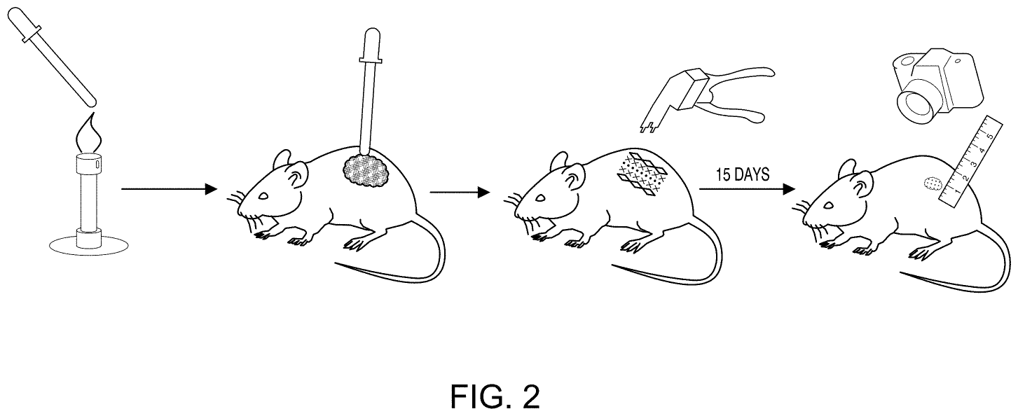

FIG. 2 shows the healing property of the formulations. For this purpose, each formulation (film) was applied on a wound of a BALB^{c} mouse and examined at day 5, 10 and 15.

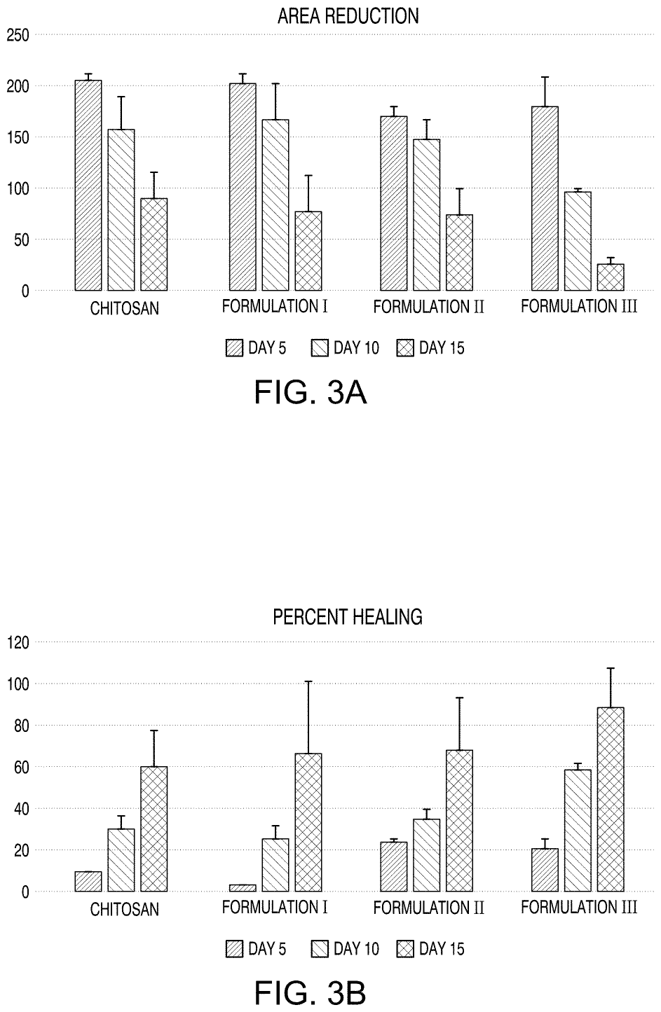

Similarly, FIGS. 3A and 3B demonstrate quantitatively that formulations I, II and III have positive effects on healing. Among these, formulation III (chitosan-Cu-MMT) treatment enhanced more wound closure with approximately 80% percent healing at the 15^{th} day.

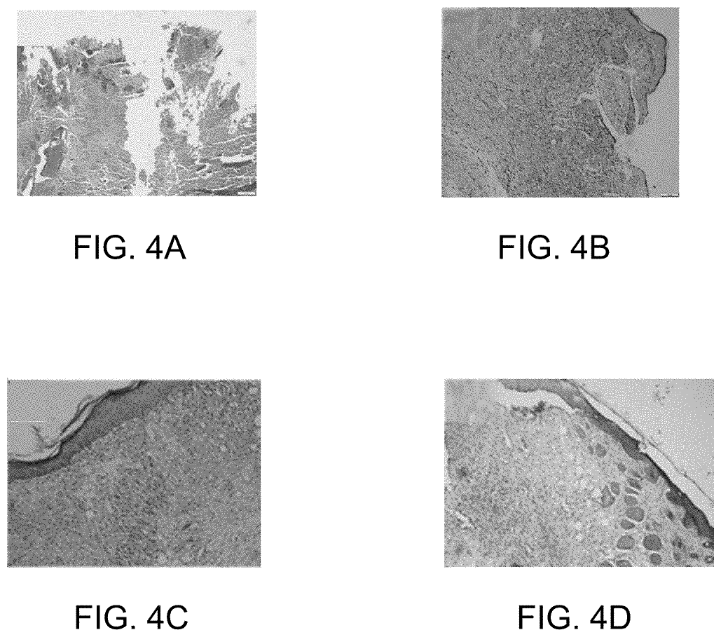

Furthermore, to evaluate the tissue regenerative property of chitosan and its formulations, histological analysis was performed. For this, formalin stored tissue sample was first dehydrated in increasing concentrations of alcohol (70%, 80% and 100%) followed by fixation in paraffin wax blocks. These blocks were sliced using a microtome and stained with hematoxylin and eosin (H&E) dye. Stained tissues were adjusted on a slide and examined under a microscope. FIGS. 4A-4D show photographs of H&E stained tissue treated with chitosan alone (FIG. 4A), formulation I (FIG. 4B), formulation II (FIG. 4C), and formulation III (FIG. 4D). FIGS. 4A-4D show that formulation III (chitosan-Cu-MMT) showed excellent new skin tissue formation, while formulation II (chitosan-Ca-MMT) and I (chitosan-Na-MMT) have good tissue regeneration capabilities. However, chitosan films showed limited tissue regenerative potential.

What is claimed is:

1. A formulation comprising chitosan impregnated with a modified clay, wherein the modified clay is a smectite clay modified with cations.

2. The formulation of claim 1, wherein the smectite clay is montmorillonite.

3. The formulation of claim 1, wherein the chitosan is highly deacetylated.

4. The formulation of claim 1, wherein the chitosan is at about 90% or more deacetylated.

5. The formulation of claim 1, wherein the cations are monovalent or divalent cations.

6. The formulation of claim 1, wherein the cations are Na⁺, Ca²⁺ or Cu²⁺.

7. The formulation of claim 1, wherein the concentration of chitosan in the formulation is 1-4% w/v.

8. The formulation of claim 1, wherein the concentration of modified clay in the formulation is 1-3% w/v.

9. The formulation of claim 1 in the form of a film, a gel, nano fibers, or a scaffold.

10. A method of wound healing comprising applying the formulation of claim 1 to a wound.
